(19) United States
(12) Patent Application Publication
(10) Pub. No.: US 2025/0185696 A1
(43) Pub. Date: Jun. 12, 2025

(54) NOVEL MENAQUINONE-N (VITAMIN-K2-7) SUPPLEMENTATION FOR LIVESTOCK AND POULTRY

(71) Applicant: SYNERGIA LIFE SCIENCES PVT LTD., Mumbai (IN)

(72) Inventors: Dilip MEHTA, Mumbai (IN); Anselm DESOUZA, Mumbai (IN)

(73) Assignee: Docketing@novozymes.com, Bagsvaerd (DK)

(21) Appl. No.: 18/844,902

(22) PCT Filed: Mar. 8, 2023
(86) PCT No.: PCT/IB2023/052181

§ 371 Date: Sep. 6, 2024

(30) Foreign Application Priority Data
Mar. 9, 2022 (IN) 202221012704

(51) Int. Cl. A23K 20/174; A23K 50/75; A61K 31/122

(52) U.S. Cl. CPC A23K 20/174; A23K 50/75; A61K 31/122

(57) ABSTRACT

The present invention relates to novel animal feed supplements that comprise menaquinone-n, wherein (n) is the number of repeating 5 carbon units. The supplements further comprise saccharides, carotenoids, xanthophylls, minerals and electrolytes. The animal feed supplement comprises menaquinone-n, wherein (n) is the number of repeating 5 carbon units, enhances and the growth, reduces the mortality, and improves the feed conversion ratio, improves liver and bone health, and replaces the need for use of probiotics in livestock and/or poultry, especially monogastric livestock and/or poultry receiving such animal feed supplements.

### RELATED APPLICATION

This application is related to and takes priority from the Indian Provisional Application 202221012704 filed Mar. 9, 2022 (9/3/2022) and is incorporated herein in its entirety.

### FIELD OF THE INVENTION

The present invention relates to methods of improving growth, reduces mortality, improved liver and bone health, and improves feed conversion ratio in livestock and poultry and animal feed supplements for achieving the same.

### BACKGROUND OF THE INVENTION

It is important that poultry and livestock achieve rapid growth and body weight gain so that they may be shipped earliest for markets. Activities such as general handling, debeaking, tagging, medications, vaccinations, transportation, any kind of nutritional inadequacy, water shortage etc. result in a great deal of stress in birds and livestock. In addition, feeding and rearing of poultry and livestock are often carried out in intensive farming; consequently, poultry and livestock may become greatly susceptible to contracting and transmitting infections. Broiler chickens have the genetic potential for significant weight gain in a short period of time, if right nutrition is provided, right from day one. If proper nutrition is provided, the growth rate of the bird has the potential to achieve a body weight of 2800 g within 42 days. Poultry industry has developed into a well-organized sector in the past three decades, especially in developing countries, thereby meeting protein needs of human body at a comparatively lower cost. Poultry industry is one of the fastest growing segments of the agricultural sector. The global poultry market is expected to grow from $310.7 billion in 2020 to $322.55 billion in 2021 at a compound annual growth rate (CAGR) of 3.8%. The market is expected to reach $422.97 billion in 2025 at a CAGR of 7% (https://www.prnewswire.com/news-releases/global-poultry-market-report-2021-301232418.html). Poultry meat is an excellent source of high quality protein, vitamins and minerals. Broiler meat is rich in nutrients and is primarily a source of high protein (21.4 g/100 g) with fewer calories (119 kcal/100 g) (Chauhan et al. 2020).

Further, productivity of a poultry and livestock largely depends on the food conversion ratio (FCR), i.e., the amount of fodder required to achieve a certain amount of product (poultry or livestock meat). It has been previously shown that improving the nutrition quality of the feed increased poultry growth rate in modern strains. The increased growth is reflected in either an increased weight of the adult chicken, or a reduction in the period of time required for obtaining an adult chicken. Current global trend is to reduce the carbon footprint for rearing livestock. Supplementation of MK 2-7 enhances meat production with lower feed.

A number of methods have been provided to achieve this end, among them are:

Materials promoting growth, the so-called “growth stimulants”, are typically employed in animal feed for producing quicker growth and increased meat tissue production. Known growth promoting materials may be categorized as probiotics, synthetic chemical growth promoters, or hormones, particularly sexual hormones. For example, U.S. Pat. No. 4,929,600 discloses a method for improving the carcass quality of poultry, via manipulation of the hormone system of the poultry including altering blood levels of hormones in the bodies of poultry by adding effective amounts of metabolically-active thyroid hormone. U.S. Pat. No. 5,017,560 discloses the administration of porcine prolactin (pPRL) to poultry in dosages of from about 1-500 μg/kg/day, preferably 10-100 μg/kg/day, to promote growth by improving the rate of weight gain and/or increasing feed utilization efficiency. Mineral feed supplements for poultry have also been disclosed as a means to improve weight gain. U.S. Pat. No. 5,459,162 discloses a copper feed supplement for chickens and method of using same. U.S. Pat. No. 7,045,150 discloses a nutrient formulation containing tellurium for use in poultry, and a method of feeding it which improves subsequent viability, cumulative feed efficacy or weight gain. U.S. Pat. No. 5,985,336 discloses nutrient formulation including moisture which is designed for use in poultry and other animals, and a method of feeding it which improves subsequent viability, cumulative feed efficiency and weight gain. The method comprises making available for consumption ad libitum a high moisture material containing at least about 20% by weight water to the poultry or other animals before they are offered dry food ad libitum. U.S. Pat. No. 6,258,399 discloses a composition containing monosaccharides, disaccharides, oligosaccharides fed immediately after hatching and during the first days of life that has a growth enhancing and mortality reducing effect. International Application Publication No. WO 2007/015932 relates to supplementing a poultry diet with phosphorylated glucomannan polysaccharides to reach increased rate of poultry weight gain, more efficient feed-to-gain and increased size of the poultry breast meat. U.S. Patent application Publication No. 20030099624 discloses the administration of spores or live cells of Bacillus laterosporus to poultry, which cause one or more of: (i) weight gain; (ii) increased feed conversion to mass; and (iii) decreased mortality. International Application Publication No. WO 01/87085 relates to animal feed, particularly poultry or pig fed suitable for feeding at the animal's early life stage including a fungi chosen from a genera within the Basidiomycetes, an extract, a derivative or a mixture thereof, and use thereof for the improvement of weight gain and/or for reducing the feed conversion and/or for the improvement of the feed value and/or health and well-being of the animal. Bioactive compounds naturally present in unprocessed milk and eggs have been shown to have a positive effect on developmental, immunological and nutritional aspects in several human and commercially viable livestock. Among them insulin has been suggested as one of the trophic factors present in colostrum. The concentration of insulin in human and pig colostrum is 3-30 fold greater than that in the serum. Insulin has been shown both in vitro and in vivo to accelerate a number of gastrointestinal (GI) functions. Oral intake of insulin by newborn pigs and rats has been shown to enhance development of intestinal activity and growth. Enteral administration of insulin was found to be beneficial in reducing food intolerance in human preterm infant (Shulman R J 1990. Pediatr Res 28:171-175; Shulman R J 2002. Arch Dis Child Fetal Neonatal Ed 86:F131-F133). International (PCT) Patent Application Publication No. WO 2005/115473 to some of the inventors of the present invention and co-workers showed that insulin and insulin derivatives provided to chicks through drinking water or feed resulted in a higher weight gain of the chicks compared to those received same feed without insulin. However, to achieve these results high amounts of insulin and at least 21 days of insulin administration were required.

Calcium (Ca) is an essential mineral for poultry. It is a main component of bone tissue and eggshell production and is used in acid-base balance and enzymatic system. Pelicia et al. (2009) showed that each egg contains about 2.2 g of calcium, mainly in the eggshell. Moreover, calcium deficiency can result in skeletal deformation, rickets, tibial dyschondroplasia, bone fracture, neural weakness, and poor feather condition in poultry. The calcium source can also affect eggshell quality, bone mineralization and growth (Brister et al. 1981; Guinotte and Nys 1991; Keshavarz et al. 1993). Apart from a good Ca source, better Ca utilization and metabolism is equally important. Despite generous supply with Ca along with other nutrients via complete diets non-infectious skeletal disorders are frequently observed in conventional broiler fattening. These incidences suggest that poultry may be faced with a condition known as “Calcium Paradox” as calcium is not getting effectively deposited in the bones leading to non-infectious skeletal disorders instead it may be getting increasingly deposited in the arteries leading to cardiovascular events. In a study by Düngelhoef et al. (2014), the extra supply of Vitamin D3 and/or Calcium along with phosphorus in fast growing chickens via drinking water in addition to these nutrients already generously included in the diet was of no benefit in solving the problem of non-infectious skeletal disorders. Furthermore, there were no effects on growth performance, blood parameters and bone mineralization (Düngelhoef et al. 2014). It was proposed that some other biochemical processes might be involved in calcium utilization. Yet another phenomenon called ‘sudden death syndrome’ has been reported where intensive broiler farming has been practiced. Those affected die suddenly, with a short, terminal, wing-beating convulsion (Meshram and Bijoy 2017). It was found to be a metabolic disorder that predisposes birds to cardiac arrhythmia. The phenomenon was observed in healthy, fast growing broiler chickens in commercial poultry farms. It was thought stress is most likely trigger of cardiac arrhythmia in broilers (Olkowski et al. 2007). The incidence of ‘sudden death syndrome’ can be minimized by slowing the growth rate of broilers, particularly during first three weeks of growth by modulating nutrient intake. However, initial three weeks are crucial to achieve bird weight gain.

It is important that poultry and livestock achieve rapid growth and body weight gain so that they can be shipped at the earliest to the markets. The rapid spread of infections amongst the poultry and livestock in the feeding facility can be caused due to feed contamination by fecal droppings. Hence there is a need for feed supplementation which will increase the rate of growth in poultry and livestock and facilitate more efficient utilization of feed, while reducing or replacing the need to use probiotics.

There have been some feed supplements and components of feed supplements that have been studied for their related on poultry and livestock. US patent U.S. Pat. No. 5,928,686 and the PCT application WO96/39862 describes a process for enhancing the health, livability and cumulative weight gain of poultry by making available for consumption a high moisture solid containing 30% to 90% by weight water and 10 to 70% dry matter, and wherein the dry matter contains at least 10% carbohydrate, and between 15 to 50% by weight of an amino acid source. British Poultry Science, 2004 October; 45 (5): 684-94 describes effects of mushroom and herb polysaccharides on growth performance of broilers. Berlin Munch Tierztl Wochenschr. 1999 October-November; 112 (10-11): 370-9 is an article on antibiotic growth promoter effects on animal nutrition.

British Poultry Science 1994 March; 35(1): 123-33, describes the effects of antibiotic growth promoters on monensin toxicity in broiler chicks. Nutrition Journal 2004, 3:19 reviews the anti-cancer effects of various antioxidants and probiotics. U.S. Pat. No. 6,558,718 describes nutrient clusters for food products. US patent U.S. Pat. No. 4,914,135 describes the use of methylsulfonylmethane to treat parasitic infections. Patent application WO9736603A1 describes biocontrol agents for use in the treatment of opportunistic infection. International Journal of Poultry Science 3 (9): 603-607, 2004 is a paper that evaluates the inhibition of pathogenic microbial growth on chicken feed by Lactobacilli. MSM: The Definitive Guide, Authors Jacob, S. W and Appleton, J, Freedom Press is a review of the science of methylsulfonylmethane. Poultry Science, 2004 M; 83 (7): 1148-54 is an article that describes the effects of mannan oligosaccharide, bacitracin methylene disalicylate on the live performance and intestinal microbiology of turkeys. J Nutrition 2004 June; 134 (6): 1487-92 is a paper that describes the role of dietary probiotics in enhancing the bioavailability of alpha-tocopheryl acetate in broilers. Infection 1999; 27 Suppl 2: S35-8 is a review antibiotic containing feed additives. However, most of the feed supplements, especially those of poultry and livestock include probiotics as one of their components.

Additionally, in recent years, the public awareness regarding the safety of food products, particularly fresh produce has been raised significantly. There is a growing demand for poultry and other meat products which are free of growth promoters, particularly probiotics and hormones. However, as described hereinabove, growth stimulants increase the food conversion ratio and the economic value of a poultry flock. Designing and using an efficient feed is important for the proper growth, reducing mortality and improving the feed conversion ratio of the livestock or poultry. Feeds-contain sources of energy, protein, essential fatty acids, minerals, essential amino acids and vitamins such as vitamin C and E. In addition, a feed also contains additives/supplements in small proportion. Recently, feed additives or supplements are gaining importance in the poultry industry because of their role in the growth of healthy birds (Abd El-Hack et al. 2020). Feed additives impart a wide spectrum of beneficial effects such as growth promotion, immune enhancement, reduction of stress, etc. Feed additives are generally considered as substances that are used to enhance the effectiveness of nutrients and exert their effects on improving poultry performance (Ashour et al. 2020). There are a number of feed additives used in poultry feed, such as probiotics, probiotics, oligosaccharides, enzymes and organic acids (Bin-Jumah et al. 2020). They are included in the diet of poultry and animals for promoting growth through their potential effect in increasing feed intake (Demir et al. 2003). An optimal feed additive in poultry feed can contribute to an increase in the production of poultry protein for human consumption, increase in body weight, which in some instances can decrease the cost of animal and poultry production and decrease in the rate of mortality (Alagawany et al. 2016). Consequently, there remains a need for feed supplements or additives that accomplish the same.

The present invention describes novel supplements that replace the use of probiotics in poultry and livestock feeds and addresses the needs of the livestock and poultry industry.

### SUMMARY OF THE INVENTION

The present invention successfully addresses the drawbacks of the known methods and compositions by providing feed supplements that comprise menaquinone-n, wherein (n) is the number of repeating 5 carbon unit and methods for enhancing and the growth, reducing the mortality, improving liver and bone health, replacing the use of probiotics in animal feed and supplements, and improving the feed conversion ratio in livestock and poultry by providing such feed supplements to livestock and poultry.

The present invention is based on the unexpected discovery that Menaquinone-n, wherein (n) is the number of repeating 5 carbon units, vitamers, or combinations thereof, as animal feed supplements, enhances growth promotion, improves liver and bone health, replaces the use of probiotics, improves the feed conversion ratio along with decrease in mortality rate in livestock and poultry.

Without being bound by any particular theory or mechanism of action the promotion of growth and reduction in mortality rate in the present study is probably due to Menaquinone-n's role in reducing arrhythmia and cardiac arrest associated with intensive farming, by improved circulation. Further, it is also hypothesized that Menaquinone-n could promote healthy liver function and overall improves the FCR.

Moreover, the teachings of the instant invention are advantageous over previously known methods as it enhances and promotes (i) increase in body weight; (ii) good feed conversion ratio (FCR); (iii) reduces the mortality of immature and developing animals, (iv) improves the livestock cost efficiency, (v) improves liver and bone health, (vi) and replaces the use of probiotics in livestock and poultry.

An aspect of the invention relates to a method of enhancing the weight gain in livestock and poultry comprising feeding livestock and poultry feed supplement comprising menaquinone-n, wherein (n) is the number of repeating 5-carbon units, a vitamer, or combinations thereof, in an amount effective to enhance the weight gain in livestock and poultry.

A further aspect of the invention relates to a method of improving the efficiency of animal feed utilization in livestock and poultry comprising feeding livestock or poultry and feed supplement comprising menaquinone-n, wherein (n) is the number of repeating 5-carbon units, a vitamer, or combinations thereof, in an amount effective to improve the efficiency of animal feed utilization in livestock and poultry.

Another aspect of the invention relates to a method of reducing the mortality in livestock and poultry comprising feeding livestock or poultry a feed supplement comprising menaquinone-n, wherein (n) is the number of repeating 5-carbon units, a vitamer, or combinations thereof, in an amount effective to reduce the mortality of livestock and poultry.

A further aspect of the invention relates to an animal feed or supplement comprising menaquinone-n, wherein (n) is the number of repeating 5-carbon units, a vitamer, or combinations thereof.

A further aspect of the invention relates to an animal feed supplement comprising menaquinone-n, wherein (n) is the number of repeating 5-carbon units, a vitamer, or combinations thereof.

Another aspect of the invention relates to a method of increasing the body weight of livestock and poultry comprising feeding livestock or poultry a feed supplement comprising menaquinone-n, wherein (n) is the number of repeating 5-carbon units, a vitamer, or combinations thereof, in an amount effective to increase the body weight of livestock and poultry.

A further aspect of the invention relates to a method of reducing stress induced sudden death syndrome in livestock and poultry comprising feeding livestock or poultry an animal feed supplement comprising menaquinone-n, wherein (n) is the number of repeating 5-carbon units, a vitamer, or combinations thereof, in an amount effective to reduce such stress.

Another aspect of the invention relates to a method of improving liver and/or bone health in livestock and poultry comprising feeding livestock or poultry an animal feed supplement comprising menaquinone-n, wherein (n) is the number of repeating 5-carbon units, a vitamer, or combinations thereof, in an amount effective to improve liver and/or bone health.

A further aspect of the invention relates to a replacing the use of probiotics in livestock and poultry comprising feeding livestock or poultry an animal feed supplement comprising menaquinone-n, wherein (n) is the number of repeating 5-carbon units, a vitamer, or combinations thereof, in an amount effective to replace the use of probiotics.

### BRIEF DESCRIPTION OF THE DRAWINGS

FIG. 1: Trend of body weights in both the study groups. Dose of Menaquinone-7 was as per Table-1.

FIG. 2: Comparison of Mortality rates in both the study groups from Day 0 to Day 28. Dose of Menaquinone-7 was as per Table-1

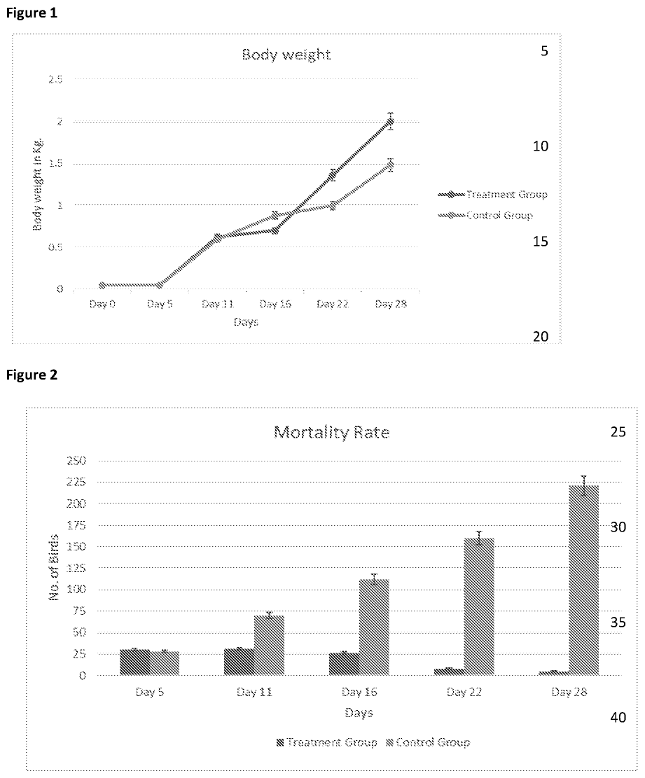

### DETAILED DESCRIPTION OF THE INVENTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

As used herein, the term “comprise” or “comprises”, or variations of the term such as “comprising” or “comprised”, are used herein to denote the inclusion of the stated integer or integers, unless in the context of usage an exclusive interpretation of the term is required.

As used herein, the term “food supplement” or “feed additives” as used herein also includes supplements usually referred to as “feed supplements”. Feed supplement refers to compositions comprising pharmaceutical, probiotic minerals or nutritional substances that are added to animal feed or drinking water simultaneously, sequentially, or serially to improve animal health and performance. Feed supplements can be from a diverse group of materials and comprise vitamins, amino acids, probiotics, antioxidants, enzymes, plant extracts, minerals, probiotics, toxin binders and other nutritional supplement elements.

As used herein, the terms “increasing” or “enhancing” (e.g., body weight) refers to at least 0.05%, 0.1%, 0.5%, 1%, 2%, 5%, 10%, 15%, 20% increase in an examined parameter of the present invention including body weight, feed efficiency, intestinal function or intestinal mass in a poultry treated with insulin as compared to non-treated poultry.

As used herein, the term “growth” or “growth performance” refers to increases in either, or both, weight and size (e.g., height, width, diameter, circumference, etc.) over that which would otherwise occur without implementation of the methods and/or administration of the compositions of the present invention. Growth can refer to an increase in the mass (e.g., weight or size) of the entire animal or of a particular tissue (e.g., muscle tissue in general or a specific muscle). Alternatively, growth can indicate a relative increase in the mass of one tissue in relation to another, in particular, an increase in muscle tissue relative to other tissues (e.g., adipose tissue). Growth further relates to nutritional status and disease resistance wherein improvement of nutritional status and/or increase in disease resistance is also indicative of improved growth performance.

As used herein, the term “about” refers to ±10%. It is required that any numeric value in the application will be referred to as if it is preceded by the term “about”.

As used herein the term “poultry” refers to domesticated fowl kept primarily for meat and eggs; including birds of the order Galliformes, e.g., the chicken, turkey, guinea fowl, pheasant, quail, and peacock; and Anserigormes (swimming birds) e.g., the duck and goose. The term also refers to pigeons, doves, or game birds like pheasants. Accordingly, the term “chick” refers to the young of the above-described birds.

As used herein, the term “immature bird” refers to a member of the avian species that lacks complete growth, differentiation, or development. Such members can have the potential capacity to attain a definite mature form or state. An immature bird can be from about 1 to about 50 days old, preferably about 1 to about 21 days old, and more preferably about 1 to about 5 days old or can have a body weight comparable to birds within these ranges.

As used herein, the term “developing bird” refers to a member of the avian species that is older or weighs more than an immature bird.

As used herein, the term “mature bird” refers to a member of the avian species that is older or weighs more than a developing bird.

As used herein, the term “broiler chick” refers to any immature chicken produced or eventually used for meat consumption.

As used herein, the term “livestock” refers to domesticated animal(s) used as a meat source. For example, cattle, sheep, goals, pigs or swine, and the like.

As used herein, the term “monogastric” refers to animal(s) having a stomach with only a single compartment. For example, poultry, pigs or swine, and the like.

As used herein, the term “pre-starter feed” refers to the feed given to animal(s) between about day 0 and about day 12 of the animal's life.

As used herein, the term “starter feed” refers to the feed given to animal(s) between about day 13 and about day 21 of the animal's life.

As used herein, the term “finisher feed” refers to the feed given to the animal(s) between about day 22 and about day of slaughter of the animal.

As used herein, the term “probiotics” refers to live microbial feed supplements that beneficially affect the host animal by improving its intestinal microbial balance. For instance Bifidobacteria has been suggested to protect against gastroenteritis associated with enteropathogenic bacteria (in particular the toxins produced by the same), as well as inhibit the growth of Salmonella, in addition such “probiotic microbes” or “friendly microbes” help in the absorption and metabolism of carbohydrates, lipids, proteins and amino acids, as well as salvage energy and vitamins from such components that have escaped digestion and absorption in the stomach and small intestine. They may also carry out detoxification and subsequent excretion of toxic factors that are either produced and/or released into the gastro-intestinal tract, provide additional uncharacterized factors, such as anti-microbial factors, and have anti-tumor/anti-carcinogenic effects. Examples of probiotics include among others Lactobacillus acidophilus and Saccharomyces bouldardii.

As used herein, the term “saccharides” refers to any carbohydrates and include to mono, di, oligo and polysaccharides. Monosaccharides are molecules that have the general formula CₙH₂ₙOₙ where n is an integer equal to 6 in case of dominant food monosaccharides. Examples of monosaccharides include glucose, fructose, galactose etc. Disaccharides are molecules that are composed of two monosaccharides units linked together by a “glycosidic” bond. Examples of disaccharides include sucrose, lactose, maltose etc. Oligosaccharides and Polysaccharides are saccharides that have more than three monosaccharide units. Examples of such oligo/polysaccharides include maltotrioses, dextrans, mannans, starch etc.

As used herein, the terms “growth” or “growth performance” refer to increases in either, or both, weight and size (e.g., height, width, diameter, circumference, etc.) over that which would otherwise occur without implementation of the methods and/or administration of the compositions of the present invention. Growth can refer to an increase in the mass (e.g., weight or size) of the entire animal or of a particular tissue (e.g., muscle tissue in general or a specific muscle). Alternatively, growth can indicate a relative increase in the mass of one tissue in relation to another, in particular, an increase in muscle tissue relative to other tissues (e.g., adipose tissue). Growth further relates to nutritional status and disease resistance wherein improvement of nutritional status and/or increase in disease resistance is also indicative of improved growth performance.

### Menaquinone-n:

Vitamin K2 is arguably one of the most interesting vitamins of this decade. It is known to have beneficial effects on bone health, cardiovascular health, muscles, oxygen uptake and the nervous system, among others, in humans. Vitamin K2 is an analogue of Vitamin K. Vitamin K is an important fat soluble vitamin. Vitamin K comprises of a group of substances, which are widespread in nature and are essential co-factors in humans in the synthesis of several proteins involved in growth and metabolism. Vitamin K has several forms which include, Vitamin-K1 (phylloquinone) that is made by plants and Vitamin K2 or MK-n (menaquinone-n, a variously sized molecule depending on the number of (n) of repeating 5-carbon units).

Unexpectedly, the present invention shows that MK-n administration for as short a period as about seven days at a cumulative amount of less than 10 μg/kg of body weight target livestock of poultry suffices to achieve the beneficial effects of increase body weight, improved liver and/or bone health, improved CFR, replacement of the use of probiotics, and reduced mortality in livestock and poultry. MK-n feed supplement should start a short time after hatching or birth, from immediately after hatching or birth to about 2-3 days post hatching or birth. In some preferred embodiments, feed supplements comprising MK-n administration starts on the first day of hatching or birth of the poultry or livestock, respectively.

The instant invention relates to Menaquinone-n, wherein (n) is the number of repeating 5-carbon units, preferably wherein (n) is up to 13 repeating units, most preferably upto 9 repeating units. The number of repeating units are selected from upto 13, upto 12, upto 11, upto 10, upto 9, upto 8, upto 7, upto 6, upto 5, upto 4, upto 3 and upto 2 carbon units. Preferably upto 9, upto 7, upto 6, upto 5, upto 4, upto 3, and upto 2 carbon units. Most preferably, having upto 7 repeating 5-carbon units, vitamers, or combinations thereof.

W. E. Lambert, A. P. De Leenheer, in Encyclopedia of Separation Science, 2000 discusses these molecules. All K-vitamers are derivatives of the same 2-methyl-1,4-napthoquinone nucleus. The number of isoprene units or the number of carbon atoms in the side chain can be used to characterize the molecules. For example, MK-4 contains four isoprene units. Menaquinone-n, also called MK-n, is characterized by a propenyl side chain often containing a large number of isoprene units (up to 13), with n indicating the number of units. Feed supplements of the present invention comprise Menaquinone-n, wherein (n) is the number of repeating carbon units, preferably repeating units representing from MK-2 to MK-13, more preferably from MK-2 to MK-7, and most preferably MK-4 to MK-7, vitamers and combinations thereof. These Menaquinones are synthesized by bacteria (e.g. Bacillus spp, Lactobacillus spp, Escherichia coli and Staphylococcus aureus). Menaquinone-n, wherein (n) is the number of repeating carbon units and the Menaquinone-n is selected from MK-2, MK-3, MK-4, MK-5, MK-6, MK-7, MK-8, MK-9, MK-10, MK-11, MK-12, and MK-13, preferably MK-2, MK-3, MK-4, MK-5, MK-6 and MK-7, more preferably MK-4, MK-5, MK 6 and MK-7, and most preferably MK-4 and MK-7, vitamers or combinations thereof.

The menaquinones mostly found in food are MK-4 (a short chain menaquinone) and the long chain menaquinones MK-7, MK-8 and MK-9. Vitamin K2 is an essential cofactor for γ-carboxylase. Compared to the other Vitamin K analogues, Vitamin K2 has the most potent γ-carboxylation activity (Faruqui et al. 2014). Due to its molecular structure, menaquinone-7 (MK-7) is more lipophilic and has much longer half-life than Vitamin K1. Regular MK-7 intake therefore results in blood levels that are not only more stable but also 7-8 times higher than when not regularly taken. Compared with Vitamin K1 distribution of MK-7 in the various tissues is significantly better. It plays important role in bone and heart health (Sato et al. 2012; Schurgers et al. 2007).

MenaquinGold® contains all natural vitamin K2 in the highly bioavailable form of Vitamin K2-7 (Menaquinone-7, MK-7). It is being manufactured by Synergia Life Sciences in a proprietary patented fermentation process. Vitamin K 2-7 is a long chained molecule that offers U.S. Pat. No. 246,985 Indian Patent Application Number 2329/MUM/2007 superior bioavailability and health effects in low doses in humans. Safety assessment of Menaquinone-7 for use in human nutrition has been successfully carried out (Ravishankar et al. 2015). MenaquinGold® is an ingredient that has Novel Food Approval (EC notified list); has self-affirmed GRAS with extensive safety; and has toxicity studies conducted to prove it safe.

Major parameters that drive the livestock and poultry business are i) increase in body weight ii) good feed conversion ratio (FCR) and iii) cost efficiency. Modern intensive poultry production has achieved phenomenal gains in the delivery of high quality and safe chicken meat, eggs and poultry bio-products. Although making gains in production and efficiency is important, the industry has to maximize the health and well-being and address the drivers of the livestock and poultry business and minimize the impact on the environment. It has been surprisingly found that employing Vitamin-K2-7 (Menaquinone-7) MenaquinGold® (brand name) in feed supplements addresses and meets these significant drivers in livestock and poultry business.

### Livestock or Poultry Diet and Administration Methods:

The particular livestock or poultry animal feeds which are useful in the present invention are not critical. A variety of livestock or poultry feeds are available commercially. Typical poultry feeds which can be useful to provide the presently claimed feed supplement to young poultry according to the teachings of the present invention are disclosed in M. 0. North and D. D. Bell, “Commercial Chick Production Manual,” Chapman & Hall, 4th Ed., 1990, and may contain mixtures of the following: carbohydrates, such as, barley; buckwheat; cassava; corn, for example, yellow corn, white corn and high-lysine corn; millet (proso); molasses; oats; rice; rye; sorghums, for example, kafir and milo; triticale; and wheat; mill by-products, such as, hominy feed; rice bran; rice hulls; wheat by-products, for example, wheat bran and wheat millings, shorts; fats and oils, such as, hard fats from slaughtered cattle; soft fats, for example, yellow grease; hydrolyzed animal fats; vegetable oils; and polyunsaturated fatty acids in egg yolks; proteins of animal origin, such as, dried blood; dried poultry waste, for example, dried cage layer manure; liver meal; meat by-products, for example, meat scraps and meat and bone meal; milk products, such as, dried skim milk; dried butter milk, and dried whey; poultry by-products, such as, hydrolyzed poultry feather meal; poultry hatchery by-product meal, for example, eggshells, unhatched and infertile eggs, and culled chicks; proteins of fish origin, such as, white fish meal; dark fish meal; and shrimp meal; proteins of vegetable origin, such as, corn gluten, coconut (copra) oil meal; cottonseed meal; guar meal; linseed (flax) oil meal; peanut (groundnut) meal; rapeseed oil meal (canola meal); safflower meal; sesame meal; soybean meal; full-fat soybeans; and sunflower seed meal; green leafy products, such as, alfalfa products, for example, sun-cured alfalfa meal, dehydrated alfalfa meal, and dehydrated alfalfa leaf meal; macrominerals, such as, curacau (island) rock phosphate (CaHPO₄)(CaHPO₄H₂O); dicalcium phosphate (CaHPO₄2H₂O); rock phosphate; steamed bone meal (Ca₃(PO₄)₂); argonite (CaCO₃); limestone (CaCO₃); oyster shell (CaCO₃); gypsum (CaSO₄2H₂O) and salt (NaCl); and vitamins, minerals and trace ingredient, such as, fat-soluble vitamins, for example, vitamins A, D, E, and K; water-soluble vitamins, for example, C (ascorbic acid), thiamin (B₁), riboflavin (B₂), pantothenic acid, niacin, pyridoxine (B₆), choline, biotin, folacin (folic acid), B₁₂ (cobalamin); minerals, such as, calcium; phosphorus; vitamin D; sodium; chlorine; potassium; sulfur; iodine; fluorine; iron; copper; manganese; magnesium; selenium; vanadium; and zinc; amino acids, such as, methionine; cystine; lysine; tryptophan; and arginine; and other feed constituents, such as, probiotics; arsenicals; xanthophylls; antioxidants; coccidiostat; electrolytes; pellet binders; tranquilizers and other supplements, for example, flavoring agents, enzymes, thyroactive compounds, and drugs.

The livestock or poultry feed supplement of the present invention can be a feed supplement which includes sources of protein, for example, soybean meal, fish meal, blood meal, poultry by-product (ground poultry offal), meat meal, wheat-meal, rapeseed, canola and combinations of the same. The feed supplement further includes carbohydrates, for example, corn, oats, barley, sorghum, or combinations of the same that can be ground into a meal for use in the animal feed. Additionally, the feed supplement can include supplements such as vitamins, minerals, fat, probiotics, and other substances or compounds as necessary or desired. Non-limiting examples of feed supplement of livestock or poultry diets include cereal-based feeds including cereals such as barley, corn, soya, wheat, triticale, and rye. Corn-soybean, wheat-soybean, and wheat-corn-soybean, sorghum-soybean, and corn-sorghum-soybean represent other non-limiting examples of suitable animal feeds or supplements according to the present invention. When the livestock or poultry diet of feed supplement of the present invention is a corn-soybean meal feed, the corn-soybean meal feed comprises from about 60 to about 70% corn by weight and from about 20 to about 30% soybean by weight.

The livestock or poultry feed supplement of the present invention can further be categorized as a starter diet, a grower-type diet, or a finisher-type diet. For example, in poultry, current growth regimes comprise particular formula for each growth stage, including pre-starter diet typically provided from the first day of hatching until the seventh day; starter provided until day 17, grower through day 27; and finisher feed until marketing, between 35-42 days after hatching. The precise composition and physical characteristics of the animal feed, and thus the livestock or poultry diet, will depend upon the species for which the feed is intended, the age and/or weight of the animal, and the duration of feeding, and can be readily determined by those skilled in the art. For example, according to some embodiments, feed supplements of the instant invention are administered from about the first day after hatching until about 7 days after hatching. According to other embodiments, the feed supplements of the instant invention, administration is stopped at least 7 days before marketing, or 14 days, or 21 days, or 28 days before marketing.

The livestock or poultry feed supplement of the present invention can be administered orally, either through drinking water or mixed in the feed before pellets or through intramuscular injection.

The livestock or poultry feed supplement of the present invention may be incorporated into the livestock or poultry feed or added to the feed as a supplement.

The livestock or poultry feed supplement of the present invention may be added with any of starter feed, middle feed, or finish feed, preferably starter feed and middle feed, most preferably starter feed.

### Feed Supplement Compositions:

Feed supplement compositions of the present invention preferably include one or more vitamins, xanthophylls, carotenoids and minerals.

The vitamins in the present invention may optionally include niacinamide, dl-α-tocopheryl acetate, dl-α-tocopherol (vitamin E), pyridoxine HCl, β-carotene (precursor of Vitamin A), riboflavin (vitamin B2), cyanocobalmin (vitamin B12), vitamin A, folic acid, thiamin HCl (vitamin B1), menadione (vitamin K) and ergocalciferol (vitamin D2 and D3), Ascrobic acid (Vitamin C). Ascorbic acid in particular acts as an anti-oxidant and also enhances the absorption of methylsulfonylmethane.

Carotenoids and Xanthophylls. Marigold Oleoresin contains carotenoids and zanthophylls and can be used in the supplement as the source of the same.

### Minerals and Electrolytes

The feed supplement compositions of the present invention also comprises of Sodium, Potassium, Calcium, Chloride, Phosphorous and Sulfur.

The feed supplement compositions of the present invention may be encapsulated within encapsulating material comprising polysaccharides, maltodextrin, milk powder, whey protein. Lipid, gum Arabic, vitamins or microcrystalline cellulose.

Typically, animal feed supplements are provided to livestock or poultry mixed with its regular animal feed formula or drinking water.

In some embodiments, the feed supplement compositions are in the form of a dry powder or liquid compositions.

The feed supplements and methods described in the present invention are further exemplified below. The following examples serve to illustrate the present invention. It is to be understood; however, that the examples in no way constitute a limitation on the scope of the present invention.

In some embodiments, the present invention relates to an animal feed supplement comprising MK-n, wherein (n) is the number of repeating 5 carbon units selected from 2, 3, 4, 5, 6, 7, 8, 9, 10, 11, 12 or 13, vitamers, or combinations thereof.

The animal feed supplement provided by the present invention can be mixed directly with the animal feed, such as one comprising barley, to prepare the final feed. Alternatively, the animal feed supplement can be mixed with one or more other animal feed supplements such as a vitamin animal feed supplement, a mineral animal feed supplement and an amino acid animal feed supplement. The resulting animal feed supplement including several different types of components may then be mixed in an appropriate amount with the animal feed.

The animal feed of the present invention comprises MK-n, wherein (n) is the number of repeating 5 carbon units selected from 2, 3, 4, 5, 6, 7, 8, 9, 10, 11, 12 or 13, vitamers, or combinations thereof, in an amount at least sufficient to achieve the intended effect, wherein the upper limit to the amount of MK-n, wherein (n) is the number of repeating 5 carbon units selected from 2, 3, 4, 5, 6, 7, 8, 9, 10, 11, 12 or 13, vitamers, or combinations thereof, can be determined based upon achieving the intended effect. Intended effects include, but are not limited to, enhancing animal growth and/or performance, such as weight gain, improving the efficiency of feed utilization, improving liver and/or bone health, replacing the use of probiotics, and decreasing mortality. The animal feed supplement added to the animal feed can comprise up to 100% MK-n, wherein (n) is the number of repeating 5 carbon units selected from 2, 3, 4, 5, 6, 7, 8, 9, 10, 11, 12 or 13, vitamers, or combinations thereof, by weight. The animal feed comprising the supplement comprises from about 5% to about 25% MK-n, wherein (n) is the number of repeating 5 carbon units selected from 2, 3, 4, 5, 6, 7, 8, 9, 10, 11, 12 or 13, vitamers, or combinations thereof, by weight.

Any animal is a suitable subject for the present invention, including cows, sheep, pigs, cats, dogs, ferrets, and avians, however, the present invention is preferably employed with monogastric animals. Suitable subjects can be of any age range including neonatal animals, developing animals, and mature animals. In some embodiments, the suitable subject can be an avian, preferably a chicken, and more preferably a broiler chick. In other embodiments the suitable subject can be a chicken. In still other embodiments, the suitable subject can be an immature, developing, or mature bird. In other embodiments, the suitable subject can be a chicken that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 days old, or within any range of these numbers. Thus, the present invention provides a supplement to a variety of different feeds, including pet feed, poultry feed, and pig feed.

### Dosing Regimen:

The livestock or poultry feed supplement of the present invention may be administered with a feed dose from about 0.01 to about 50 micrograms/day, preferably from about 1 to 40 microgram/day, most preferably from about 10 to about 35 microgram/day per livestock or poultry.

The livestock or poultry feed supplement of the present invention may be administered with a feed dose or effective dose from about 0.01 to about 50 micrograms/day, preferably from about 1 to 40 microgram/day and preferably from about 10 to about 35 microgram/day, and most preferably from about 3 micrograms/day to about 5 micrograms/day per livestock or poultry.

The livestock or poultry feed supplement of the present invention may be administered as a cumulative total dose of livestock or poultry feed supplement is from about 0.01 to about 50 micrograms/day, preferably from about 1 to 40 microgram/day, most preferably from about 10 to about 35 microgram/day per livestock or poultry for about 30 days.

The livestock or poultry feed supplement of the present invention may be administered to livestock or poultry from about 0 days of hatching to about 40 days, preferably 1 days of hatching to about 30 days, most preferably from about 2 days of hatching to about 30 days per livestock or poultry.

The livestock or poultry feed supplement of the present invention may be administered for at least about 40 days, preferably at least about 35 days, most preferably at least about 30 days per livestock or poultry before marketing.

Sample dosing regimen of the livestock or poultry feed supplement of the present invention may be administered in accordance with the table below.

| | |
|---|---|
| Days from birth or | Dosing per day per |
| hatching | livestock/poultry |

| | | | |
|---|---|---|---|
| 0-11 | Days | 10 | μg |
| 12-18 | Days | 15 | μg |
| 19-25 | Days | 20 | μg |
| 25-35 | Days | 35 | μg |

### Method of Use

Embodiments of the present invention further relate to methods of improving the efficiency of feed utilization of an animal feed in livestock and/or poultry comprising feeding livestock and/or poultry an animal feed supplement, where the animal feed supplement comprises MK-n, wherein (n) is the number of repeating 5 carbon units selected from 2, 3, 4, 5, 6, 7, 8, 9, 10, 11, 12 or 13, vitamers, or combinations thereof, in an amount effective to improve the efficiency of feed utilization of an animal feed provided to livestock and/or poultry. The animal feed can include the animal feeds as described above and, in particular embodiments can be corn-soybean meal.

Improving the efficiency of feed utilization refers to a reduction in the Feed Conversion Ratio (FCR) as compared with that which would otherwise occur without implementation of the methods and/or administration of the compositions of the present invention. The FCR is the ratio of the amount of feed consumed relative to the weight gain of an animal. In one embodiment of the present invention, the improved efficiency of feed utilization can occur by increasing gastrointestinal nutrient absorption without a concomitant increase in intestinal energy expenditure. The animal feed can include the animal feeds as described above and, in particular embodiments, can be corn-soybean meal.

In other embodiments, the present invention relates to methods of reducing mortality in livestock and/or poultry comprising feeding livestock and/or poultry an animal feed supplement comprising MK-n, wherein (n) is the number of repeating 5 carbon units selected from 2, 3, 4, 5, 6, 7, 8, 9, 10, 11, 12 or 13, vitamers, or combinations thereof, in an amount effective to reduce the mortality of livestock and/or poultry, for example immature birds, and more specifically, broiler chicks. The animal feed can include the animal feeds as described above and, in particular embodiments, can be corn-soybean meal. Reducing mortality refers to increasing the survivability or decreasing the death rate in animals after birth or hatch as compared with that which would otherwise occur in the absence of implementation of the methods and/or administration of the compositions of the present invention. Mortality can be from any cause, in particular, stress, stunting, “starveouts”, and disease. In some embodiments, the present invention reduces mortality in immature birds. In other embodiments, the birds are about 1, 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, or 35 days old, preferably about 1 to about 21 days old, and more preferably about 1 to about 5 days old.

In other embodiments, the present invention relates to methods of enhancing or increasing the growth or body weight of livestock and/or poultry comprising feeding livestock and/or poultry an animal feed supplement comprising MK-n, wherein (n) is the number of repeating 5 carbon units selected from 2, 3, 4, 5, 6, 7, 8, 9, 10, 11, 12 or 13, vitamers, or combinations thereof, in an amount effective to enhance or increase the growth or body weight of livestock and/or poultry, for example immature birds, and more specifically, broiler chicks. The animal feed can include the animal feeds as described above and, in particular embodiments, can be corn-soybean meal. In some embodiments, the increase or enhancement of body weight is at least upto 0.05%, 0.1%, 0.5%, 1%, 2%, 5%, 10%, 15%, or 20%, preferably at least upto 5%, 10% or 15%, most preferably at least from about 2% to about 5%.

In other embodiments, the present invention relates to methods of improving liver and/or bone health of livestock and/or poultry comprising feeding livestock and/or poultry an animal feed supplement comprising MK-n, wherein (n) is the number of repeating 5 carbon units selected from 2, 3, 4, 5, 6, 7, 8, 9, 10, 11, 12 or 13, vitamers, or combinations thereof, in an amount effective to improve liver and/or bone health of livestock and/or poultry, for example immature birds, and more specifically, broiler chicks. The animal feed can include the animal feeds as described above. In some embodiments, the improvement of liver and/or bone health is at least 0.05%, 0.1%, 0.5%, 1%, 2%, 5%, 10%, 15%, 20%, preferably at least 5%, 10% or 15%, most preferably 5% or 10%

In other embodiments, the present invention relates to methods of replacing the use of probiotics in livestock and/or poultry comprising feeding livestock and/or poultry an animal feed supplement comprising MK-n, wherein (n) is the number of repeating 5 carbon units selected from 2, 3, 4, 5, 6, 7, 8, 9, 10, 11, 12 or 13, vitamers, or combinations thereof, in an amount effective to replace the use of probiotics in livestock and/or poultry, for example immature birds, and more specifically, broiler chicks. The animal feed can include the animal feeds as described above and, in particular embodiments, crude protein and carbohydrates can be corn-soybean meal.

The objects, advantages, and novel features of the present invention will become apparent to one of ordinary skill in the art upon examination of the following examples, which are not intended to be limiting. The examples serve to illustrate the present invention. It is to be understood however, that the examples in no way constitute a limitation on the scope of the present invention.

### EXAMPLES

### Example 1

COMPOSITION OF BOILER STARTER DIET NO. 1

| Ingredients | Qty (Kg per 100 Kg) |
|---|---|
| Maize | 44.25 |
| Rice Polish | 10.00 |
| Groundnut Cake | 14.00 |
| Sunflower Cake | 15.00 |
| Fish Meal (43% protein) | 6.00 |
| Meat Meal (56% protein) | 6.00 |
| L-lysine HCI | 0.15 |
| Animal fat | 2.00 |
| Bone meal | 0.75 |
| Lime stone | 0.50 |
| Salt | 0.25 |
| Mineral and Vitamin Mixture² | 0.10 |
| Total | 100.00 |

1. Mineral mixture to contain: Ferrous sulphate, 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF GROWER DIET NO. 1

| Ingredients | Qty (Kg per 100 Kg) |
|---|---|
| Maize | 27.00 |
| Rice Polish | 40.00 |
| Wheat bran | 15.00 |
| Sunflower | 10.00 |
| Fish Meal (43% protein) | 6.00 |
| Bone meal | 0.60 |
| Lime stone | 0.80 |
| Iodized Salt | 0.40 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF BOILER FINISHER DIET NO. 1

| Ingredients | Qty (Kg per 100 Kg) |
|---|---|
| Maize | 44.10 |
| Rice Polish | 20.00 |
| Ground nut cake | 11.00 |
| Sunflower cake | 11.00 |
| Fish meal(43% protein) | 5.50 |
| Meat meal (56% protein) | 5.50 |
| Fat | 1.25 |
| Bone meal | 0.60 |
| Lime stone | 0.70 |
| Salt | 0.25 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

### Example 2

COMPOSITION OF BOILER STARTER DIET NO. 2

| Ingredients | Qty (Kg per 100 Kg) |
|---|---|
| Maize | 43.75 |
| Rice Polish | 10.00 |
| Groundnut cake | 14.00 |
| Sunflower cake | 14.00 |
| Fish meal (43% protein) | 10.00 |
| Blood meal (73% protein) | 3.50 |
| Animal fat | 3.00 |
| Bone meal | 1.15 |
| Salt | 0.50 |
| Mineral and Vitamin Mixture² | 0.10 |
| Total | 100.00 |

1. Mineral mixture to contain: Ferrous sulphate, 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF GROWER DIET NO. 2

| Ingredients | Qty (Kg per 100 Kg) |
|---|---|
| Bajra | 34.00 |
| Rice Polish | 40.00 |
| Wheat bran | 5.00 |
| Salseed meal (deoiled) | 5.00 |
| Maize gulten meal | 4.00 |
| Sunflower cake | 5.00 |
| Fish meal (43% protein) | 6.00 |
| Bone meal | 0.60 |
| Limestone | 0.50 |
| Iodized Salt | 0.40 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF BOILER FINISHER DIET NO. 2

| Ingredients | Qty (Kg per 100 Kg) |
|---|---|
| Maize | 45.30 |
| Rice Polish | 20.00 |
| Ground nut Cake | 9.50 |
| Sunflower Cake | 9.00 |
| Sunflower Cake | 9.00 |
| Blood Meal (73% protein) | 3.00 |
| Fat | 2.00 |
| Bone meal | 1.10 |
| Salt | 0.50 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

### Example 3

COMPOSITION OF BOILER STARTER DIET NO. 3

| Ingredients | Qty (Kg per 100 Kg) |
|---|---|
| Maize | 45.47 |
| Rice Polish | 10.00 |
| Groundnut cake | 30.00 |
| Sunflower cake | 13.00 |
| L-lysine HCI | 0.18 |
| DL - Methionine | 0.10 |
| Bone meal | 0.75 |
| Salt | 0.40 |
| Mineral and Vitamin Mixture | 0.10 |
| Total | 100.00 |

1. Mineral mixture to contain: Ferrous sulphate, 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF GROWER DIET NO. 3

| Ingredients | Qty (Kg per 100 Kg) |
|---|---|
| Maize | 25.00 |
| Jawar | 20.00 |
| Wheat bran | 13.40 |
| Tapioca flour | 20.00 |
| Groundnut cake | 10.00 |
| Fish Meal (43% protein) | 5.00 |
| Silk worm pupae meal (deoiled) | 4.00 |
| Bone meal | 2.00 |
| Iodized salt | 0.50 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

COMPOSITION OF BOILER FINISHER DIET NO. 3

| Ingredients | Qty (Kg per 100 Kg) |
|---|---|
| Maize | 46.80 |
| Rice Polish | 20.00 |
| Ground nut cake | 19.00 |
| Fish meal (43% protein) | 13.00 |
| Bone Meal | 0.70 |
| Iodized salt | 0.40 |
| Mineral and Vitamin Mixture² | 0.10 |
| Total | 100.00 |

1. Mineral mixture to contain: Ferrous sulphate, 20 g, Manganese sulphate 25 g, Zinc Sulphate 25 g; Copper sulphate 1.5 g; and Potassium iodate 100 mg.
²Vitamin mixture to provide: Vitamin A, 800,000 I.U., Vitamin D₃ 100,000 ICU, Riboflavin, 400 mg and Folic Acid 100 mg.

### Example 4

To prepare a Menaquinone (n) feed additive composition, any one of Menaquinone K2-7, vitamers or combinations thereof are blended in MCT oil & MCC powder with Tween80 to a concentration of 2000 ppm and add to drinking water or pelletized

### Example 5

A pilot study in 5605 chicks to evaluate the role of Menaquinone-n in bird weight increase, FCR and cost efficiency. The chicks were divided into Treatment group (N=2839) and Control group (N=2766). Menaquinone-7 (provided by Synergia Life Sciences as a 2000 ppm water dispersible powder) was administered to the treatment group for 30 days as per dosage outlined in Table 1. The birds were acclimatized for initial 4 days. Menaquinone-7 in divided doses was started on day 5 till day 30 and they were vaccinated on day 6. The chicks were observed for body weight and mortality.

### Study Design

5605 day old chicks were selected for the study. They were divided into Treatment group consisting of 2839 chicks, while the remaining 2766 were included in the Control group. The chicks were acclimatized for initial 4 days.

Menaquinone-7 (provided by Synergia Life Sciences as a 2000 ppm water dispersible powder) was administered daily employing standard commercial feed provided by Godrej Foods to the treatment group in daily dosages as outlined in Table 1. Menaquinone-7 in divided doses was started on day 5 till day 30 and the chicks were vaccinated on day 6. The body weight of the chicks was determined, and mortality rate observed on Day 11, Day 16, Day 22 and Day 28.

### Materials and Methods

### Selection of Breed

A poultry farm with two open shades was selected for the study. Vencobb 400 breed of broiler chicken were selected for the study. Vencobb 400 breed was selected because at breeder level it is an early maturing bird with early peak, sustained production and better feed efficiency. Vencobb 400 is known for its proven adaptability to varied climatic conditions and husbandry practices.

### Environmental Farming Conditions:

The chicks were housed in poultry sheds with tin rooting and open shade sides. Water was fed ad libitum through bell water feeders. The litter consisted of rice husk before housing the birds the cages were sanitized with hypo chloride, The chicks were provided with feed supplied from Godrej Tyson Foods, ad libitum.

### Vaccine:

The vaccines were supplied by Godrej. They were administered between 5^{th} to 10^{th} day after the chicks were received at the poultry farm. Natural day bright light cycle was followed. The cage was divided into 2 sections by a partition.

Menaquinone 7 supplementation was in a drinking water as in Table 1. Water was made up to the required level as required daily.

### Feed Conversion Ratio

Feed conversion ratio was calculated by taking the total amount of feed consumed by the bird and dividing it by the amount of weight gained.

### European Performance Efficiency Index (EPEI)

European Performance Efficiency Index (EPEI) was calculated by following formula (Karangiya et al. 2016)

$$E P E I = \frac{Average body weight ( g ) \times Livability ( \% )}{Age of flock ( days ) \times F C R} \div 10$$

### Feed Conversion Ratio

Feed conversion ratio was calculated by taking the total amount of feed consumed by the bird and dividing it by the amount of weight gained.

TABLE 1
Dosage of Menaquinone-7 (mixed in water) administered
to the treatment group of 2839 chicks

| | Menaquinone-7 | | |
|---|---|---|---|
| Day | dose per day | Menaquinone-7 | |
| Day 05 to day 11 (11 days) | 15 gm | 15 × 11 = 165 | gm |
| Day 12 to Day 16 (5 days) | 20 gm | 20 × 5 = 100 | gm |
| Day 16 to Day 22 (6 days) | 25 gm | 25 × 6 = 150 | gm |
| Day 23 to Day 26 (6 days) | 50 gm | 50 × 6 = 300 | gm |

### Results

### Cage Side Observations

Cage side observations like daily life behavior patterns, odor of litter etc. were recorded. The birds in the treatment group appeared comfortable with respect to the stress due to heat, vaccination and crowding in comparison to the control group. There was reduced ammonia odor in the litter in the treatment group as compared to the control group.

### Body Weight

Body weight was estimated by catching 5 birds in each group and determining the total weight and reporting the average at the intervals recorded in Table 2. At the end of treatment, after catching the total catching weight was recorded and the average weight determined.

TABLE 2
Average weight per chick in the study
groups from Day 0 to Day 28.

Average weight (kg)per chick

| | Treatment | Control |
|---|---|---|
| Day | Group | Group |
| Day 0 | 0.05 | 0.05 |
| Day 5 | 0.05 | 0.05 |
| Day 11 | 0.622 | 0.595 |
| Day 16 | 0.966 | 0.882 |
| Day 22 | 1.36 | 0.996 |
| Day 28 | 2.0 | 1.480 |

### Mortality

The mortality rate by the end of 28^{th} day in the treatment group was 0.8 per day and in the control group it was 10.16 per day. The comparison of the mortality the 2 group is as follows.

TABLE 3
Mortality rate of chicks in the Treatment
group and the Control group.

| | Treatment Group | | Control Group | |
|---|---|---|---|---|
| | Cumulative | Mortality | Cumulative | Mortality |
| Day | Mortality | rate/day | Mortality | rate/day |
| Day 0 | 0 | — | 0 | — |
| Day 5 | 30 | 7.5 | 28 | 7.0 |
| Day 11 | 61 | 2.8 | 70 | 6.0 |
| Day 16 | 87 | 5.2 | 112 | 10.4 |
| Day 22 | 95 | 1.3 | 160 | 8.0 |
| Day 28 | 100 | 0.8 | 221 | 10.16 |

| | | |
|---|---|---|
| Mortality % | 3.52 | 7.98 |

The birds in the treatment group had an average weight of 2 kg at the end of 28^{th} day. While in the control group, the average weight of each bird was 1.48 kg. The overall mortality at the end of 28^{th} day in the treatment group was 3.52% and in the control group it was 7.98%. An overall reduction in mortality rate by over 4% was observed.

TABLE 4
Comparative study results of mortality rates between
the control group and the treated group.

| | Control Group | Treated Group |
|---|---|---|
| No. of Birds | 2000 | 2000 |
| K2 Supplementation | 15 g of 2000 ppm | 15 g of 2000 ppm |
| Mortality | 9.7% | 1.8% |
| Avg wt./bird | 2.01 kg | 2.1560 kg |
| FCR | 0.840 | 0.728 |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

From the above data and discussion, applicants put forward the following key aspects of the invention:
The present invention provides a method of enhancing weight grain in livestock and poultry comprising feeding livestock and poultry feed supplement comprising menaquinone-n, wherein (n) is the number of repeating 5-carbon units selected from 2, 3, 4, 5, 6, 7, 8 and 9, in an amount effective to enhance the weight gain of the livestock and poultry.
In one aspect the invention provides a method of improving the efficiency of animal feed utilization in livestock and poultry comprising feeding livestock or poultry a feed supplement comprising menaquinone-n, wherein (n) is the number of repeating 5-carbon units selected from 2, 3, 4, 5, 6, 7, 8 and 9, vitamers or combinations thereof, in an amount effective to improve the efficiency of animal feed utilization in livestock and poultry.
In another aspect the invention provides a method of reducing the mortality in livestock and poultry comprising feeding livestock or poultry and feed supplement comprising menaquinone-n, wherein (n) is the number of repeating 5-carbon units selected from 2, 3, 4, 5, 6, 7, 8 and 9, vitamers or combinations thereof in an amount effective to reduce the mortality of livestock and poultry.
The invention also provides a method of replacing the use of antibiotic growth promoters (APG) in animal feed comprising feeding livestock or poultry a feed supplement comprising menaquinone-n, wherein (n) is the number of repeating 5-carbon units selected from 2, 3, 4, 5, 6, 7, 8 and 9, vitamers or combinations thereof in an amount effective to replace the use of APG in livestock or poultry.
In yet another aspect, the invention provides a feed supplement comprising menaquinone-n, wherein (n) is the number of repeating 5-carbon units selected from 2, 3, 4, 5, 6, 7, 8 and 9, vitamers or combinations thereof.
Furthermore, the invention provides an animal feed supplement comprising menaquinone-n, wherein (n) is the number of repeating 5-carbon units, selected from 2, 3, 4, 5, 6, 7, 8 and 9, vitamers or combinations thereof.
The present invention also provides a method of increasing the body weight of livestock and poultry comprising feeding livestock or poultry an feed supplement comprising menaquinone-n, wherein (n) is the number of repeating 5-carbon units feeding livestock or poultry a feed supplement comprising menaquinone-n, wherein (n) is the number of repeating 5-carbon units selected from 2, 3, 4, 5, 6, 7, 8 and 9, vitamers or combinations thereof in an amount effective to increase the body weight of livestock and poultry.
It will be understood by a person skilled in the art that the present invention provides a method of reducing stress induced sudden death syndrome in livestock and poultry comprising feeding livestock or poultry an animal feed supplement comprising menaquinone-n, wherein (n) is the number of repeating 5-carbon units feeding livestock or poultry a feed supplement comprising menaquinone-n, wherein (n) is the number of repeating 5-carbon units selected from 2, 3, 4, 5, 6, 7, 8 and 9, vitamers or combinations thereof in an amount effective to in an amount effective to reduce such stress.
Furthermore, the applicants also present a method for achieving at least one outcome selected from increase cumulative weight gain and enhanced feed conversion efficiency of poultry or reducing the mortality of livestock or poultry comprising orally administering to livestock or poultry Menaquinone-n, wherein (n) is the number of repeating 5-carbon units selected from 2, 3, 4, 5, 6, 7, 8 and 9, vitamers or combinations at a cumulative total dose of about 4 micrograms per individual livestock or poultry per day.
The plurality of outcomes herein is selected from cumulative weight gain and feed conversion efficiency of poultry.
The Menaquinone-n, is administered at a cumulative total dose of up to about 120 micrograms for up to 30 days per livestock or poultry or less.
The Menaquinone-n is administered at a cumulative total dose of from about 0.01 micrograms to about 40 micrograms per livestock or poultry.
The Menaquinone-n is administrated about immediately after hatching or birth to about 40 days after hatching.
The Menaquinone-n is administered about day 2 after hatching or birth to about 35 days after hatching or birth.
The Menaquinone-n is administered for at least 21 consecutive days.
The feed supplement is mixed with poultry or livestock feed formula. The poultry or livestock feed formula is in a form selected from the group consisting of powder, bolus, pellets and microcapsules.
The feed supplement is mixed with poultry or livestock drinking liquid. The feed supplement is dispersed into the drinking liquid of poultry or livestock.
The feed supplement is dispersed at a rate of about 10 grams per day for about 30 days.

### ADDITIONAL REFERENCES

Chauhan, S. S., Sharma, R. K., Singh, D. V., Shukla, S. K., & Palod, J. (2020). Comparative study of the effects of different feed supplements on feed conversion ratio and performance index of broiler chickens.
Pelicia, K., Garcia, E., Mori, C., Faitarone, A. B. G., Silva, A. P., Molino, A. B., Vercese, F. and Berto, D. A., 2009. Calcium levels and limestone particle size in the diet of commercial layers at the end of the first production cycle. Brazilian Journal of Poultry Science, 11(2), pp. 87-94.
Brister Jr, R. D., Linton, S. S. & Creger, C. R., 1981. Effects of dietary calcium sources and particle size on laying performance. Poult. Sci. 60, 2648-2654.
Guinotte, F. & Nys, Y. (1991). Effects of particle size and origin of calcium sources on eggshell quality and bone mineralization in egg laying hens. Poultry Science 70, 583-592.
Keshavarz, K., and S. Nakajima, 1993. Re-evaluation of calcium and phosphorus requirements of laying hens for optimum performance and eggshell quality. Poultry Sci. 72:144-153.
Düngelhoef, K., Wilkens M. R., Mrochen N., Schröder, B., Sander S. & Kamphues J., (2014). Effects of an extra supply of vitamin D, calcium and phosphorus via drinking water on the calcium and phosphorus metabolism in growing chickens fed a conventional complete diet. Poultry Archives. 78. 10.1399/eps.2014.24.
Meshram, P. V., Bijoy, F., & Kurkure, N. V. (2017). Evaluation of protective action Boerhaavia diffuse Linn and Butea frondosa koen in experimental ochratoxicosis in broilers in relation with haematological and immunological parameter. Blue Cross Book, (36), 49-55.
Olkowski, A. A. (2007). Pathophysiology of heart failure in broiler chickens: structural, biochemical, and molecular characteristics. Poultry Science, 86(5), 999-1005.
Abd El-Hack, M. E., El-Saadony, M. T., Shafi, M. E., Qattan, S. Y., Batiha, et al. (2020). Probiotics in poultry feed: A comprehensive review. Journal of animal physiology and animal nutrition, 104(6), 1835-1850.
Ashour, E. A., El-Kholy, M. S., Alagawany, M., Abd El-Hack, M. E., Mohamed, L. A., Taha, A. E., El Sheikh, A. I., Laudadio, V., & Tufarelli, V. (2020). Effect of dietary supplementation with Moringa oleifera leaves and/or seeds powder on production, egg characteristics, hatchability and blood chemistry of laying Japanese quails. Sustainability, 12(6), 2463.
Bin-Jumah, M., Abd El-Hack, M. E., Abdelnour, S. A., Hendy, Y. A., Ghanem, H. A., Alsafy, S. A., et al. (2020). Potential use of chromium to combat thermal stress in animals: A review. Science of the Total Environment, 707, 135996.
Demir, E., Sarica, S., Ozcan, M. A., & Sui Mez, M. (2003). The use of natural feed additives as alternatives for an antibiotic growth promoter in broiler diets. British Poultry Science, 44(S1), 44-45.
Alagawany, M., Abd El-Hack, M. E., Arif, M., & Ashour, E. A. (2016). Individual and combined effects of crude protein, methionine, and probiotic levels on laying hen productive performance and nitrogen pollution in the manure. Environmental Science and Pollution Research, 23, 22906-22913.
Sato, T., Schurgers, L. J. & Uenishi, K. Comparison of menaquinone-4 and menaquinone-7 bioavailability in healthy women. Nutr J 2012. 11, 93.
Schurgers L J, Teunissen K J, Hamulyák K, Knapen M H, Vik H, Vermeer C: Vitamin K-containing dietary supplements: comparison of synthetic vitamin K1 and natto-derived menaquinone-7. Blood. 2007, 109:3279-3283.
Ravishankar B, Dound Y A, Mehta D S, Ashok B K, de Souza A, Pan M-H, Ho C-T, Badmaev V, Vaidya A D B (2015) Safety assessment of menaquinone-7 for use in human nutrition. J Food Drug Anal 23(1):99-108.
A, Faruqui. Evolving role of vitamin K2-7 (Menaquinone) in Osteoporosis & cardiovascular health. International Journal of Pharmacological Research. (2014) 4(2). 43-48.

1. A method, comprising administering one or more menaquinone-n vitamers, wherein (n) represents the number of repeating 5-carbon isoprenyl units in a vitamer, and wherein (n) is selected from 2, 3, 4, 5, 6, 7, 8 and 9, to an animal.

2. The method of claim 1, wherein said one or more menaquinone-n vitamers is administered to said animal in an amount effective to enhance weight gain of said animal.

3. The method of claim 1, wherein said one or more menaquinone-n vitamers is administered to said animal in an amount effective to improve animal feed utilization of said animal.

4. The method of claim 1, wherein said one or more menaquinone-n vitamers is administered to said animal in an amount effective to reduce the mortality of said animal.

5. The method of claim 1, wherein said one or more menaquinone-n vitamers is administered to said animal in an amount effective to replace one or more antibiotic growth promoters.

6. The method of claim 1, wherein said one or more menaquinone-n vitamers is administered to said animal in an amounteffective to increase the body weight of said animal.

7. The method of claim 1, wherein said one or more menaquinone-n vitamers is administered to said animal in an amount effective to in an amount effective to reduce stress-induced sudden death syndrome in said animal.

8. The method of claim 1, wherein said one or more menaquinone-n vitamers is administered to said animal at a cumulative total dose of about 4 micrograms per day.

9. The method according to claim 8, wherein said one or more menaquinone-n vitamers is administered at a cumulative total dose of up to about 120 micrograms for up to 30 days.

10. The method according to claim 9, wherein said one or more menaquinone-n vitamers is administered at a cumulative total dose of from about 0.01 micrograms to about 40 micrograms.

11. The method according to claim 8, wherein said one or more menaquinone-n vitamers is administrated from about immediately after hatching or birth to about 40 days after hatching or birth.

12. The method of claim 8, wherein said one or more menaquinone-n vitamers is administered from about 2 days after hatching or birth to about 35 days after hatching or birth.

13. The method according to claim 8, wherein said one or more menaquinone-n vitamers is administered for at least 21 consecutive days.

14. The method according to claim 8, wherein said one or more menaquinone-n vitamers is administered to said animal as part of an animal feed formula.

15. The method of claim 14, wherein the animal feed formula is in a form selected from the group consisting of powders, boluses, pellets and microcapsules.

16. The method of claim 15, wherein the animal feed formula is administered at a rate of about 10 grams per day for about 30 days.

17. A method, comprising introducing one or more menaquinone-n vitamers, wherein (n) represents the number of repeating 5-carbon isoprenyl units in a vitamer, and wherein (n) is selected from 2, 3, 4, 5, 6, 7, 8 and 9, into an animal feed to produce a supplemented animal feed.

18. An animal feed comprising one or more exogenous menaquinone-n vitamers, wherein (n) represents the number of repeating 5-carbon isoprenyl units in a vitamer, and wherein (n) is selected from 2, 3, 4, 5, 6, 7, 8 and 9.
